(19) United States
(12) Patent Application Publication
(10) Pub. No.: US 2011/0306998 A1
(43) Pub. Date: Dec. 15, 2011

(54) TROCAR SYSTEM

(76) Inventor: STEVE LIVNEH, Amherstburg (CA)

(21) Appl. No.: 13/161,206

(22) Filed: Jun. 15, 2011

Related U.S. Application Data
(60) Provisional application No. 61/354,842, filed on Jun. 15, 2010.

(51) Int. Cl. A61B 17/34

(52) U.S. Cl. 606/185

(57) ABSTRACT

A trocar system is described. The trocar system allows for penetration into the patient's laparoscopic cavity while also allowing visualization once an imaging device (e.g., a laparoscope) is inserted into the hollow shaft of the trocar. Attached to a distal end of the trocar shaft is a tip portion, which may be comprised of a transparent material, and which allows for general visualization upon penetration into the patient's laparoscopic cavity. The tip may include a conical shape with at least one blade (e.g., preferably at least two blades) protruding longitudinally along a peripheral surface thereof. The blades may be comprised of a metallic material, e.g., preferably similar to the trocar shaft material, and may be securely attached (e.g., preferably welded) to the trocar shaft. The transparent tip may be caged by the blades and the trocar's shaft and may also be glued to the blades and trocar shaft as well. The metallic blades provide numerous advantages, such as but not limited to, assisting in the penetration through the patient's abdominal wall, while at the same time securing the conical transparent tip portion to the trocar's shaft and thus reducing the risk of breakage and/or unintended detachment therefrom.

### CROSS-REFERENCE TO RELATED APPLICATION

The instant application claims priority to U.S. Provisional Patent Application Ser. No. 61/354,842 filed Jun. 15, 2010, pending, the entire specification of which is expressly incorporated herein by reference.

### FIELD OF INVENTION

The present invention relates generally to trocar systems and more specifically to trocar systems with a transparent tip portion attached to a distal end of the trocar shaft, and methods for making the same.

### BACKGROUND OF THE INVENTION

Endoscopy, and especially laparoscopic endoscopy, has been a rapidly growing surgical practice in the past decades. Accessing the patient's laparoscopic cavity is typically done via holes, usually punctured with a sharp element referred to as a trocar. In order to penetrate the patient's laparoscopic cavity, the trocar is typically placed into a tubular element referred to as a cannula, such that the sharp end of the trocar is protruding from the cannula's distal end. The trocar end, when sharp, may puncture the abdominal wall. When a blunt trocar is used, an initial incision to the patient's skin may be required.

In practice, the surgeon typically presses the cannula/trocar tandem against the patient's abdominal wall and carefully penetrates into the insufflated laparoscopic cavity, while devoting great care in avoiding injuries to any internal organs. Until transparent tip portions were introduced, the penetration procedure was executed in an essentially “blind” fashion, relying mostly on the surgeon's skills. However, the addition of a transparent tip portion to the distal portion of the shaft of the trocar, as well as the use of an illuminated scope device (e.g., placed behind the tip portion within the hollow shaft of the trocar) has greatly increased the safety level and added a level of visualization such that undesired penetration of any internal organs has been greatly reduced. However, conventional systems and methods for manufacturing trocars with transparent tip portions have been difficult, expensive and laborious, along with producing unsatisfactory results.

Accordingly, there exists a need for new and improved trocar systems that include a transparent tip portion engaged to a distal portion of the trocar shaft, and methods for making the same.

### SUMMARY OF THE INVENTION

In accordance with the general teachings of the present invention, a trocar system is provided with a transparent tip portion. The trocar system allows for penetration into the patient's laparoscopic cavity while also allowing visualization once an imaging device (e.g., a laparoscope) is inserted into the hollow shaft of the trocar. Attached to a distal end of the trocar shaft is a tip portion, which may be comprised of a transparent material, and which allows for general visualization upon penetration into the patient's laparoscopic cavity. The tip portion may include a conical shape with at least one blade (e.g., preferably two blades) protruding longitudinally along a peripheral surface thereof. The blades may be comprised of a metallic material, e.g., preferably similar to the trocar shaft material, and may be securely attached (e.g., preferably welded) to the trocar shaft. The transparent tip may be caged by the blades and the trocar's shaft and may also be glued to the blades and trocar shaft as well. The metallic blades provide numerous advantages, such as but not limited to, assisting in the penetration through the patient's abdominal wall, while at the same time securing the conical transparent tip portion to the trocar's shaft and thus reducing the risk of breakage and/or unintended detachment therefrom.

The present invention also provides methods of securing the transparent tip portion to the trocar's shaft, e.g., using metallic blades. These arrangements allow for visualization through the trocar shaft while adding robust metallic blades and added security of the tip portion to the trocar shaft.

In accordance with one embodiment of the present invention, a trocar system is provided, comprising:

a shaft member having a bore formed at a distal end thereof;

a tip member having a proximal portion and a distal portion, the tip member having an area defining a groove extending along a peripheral surface thereof, the proximal portion being at least partially receivable in the bore; and

a cutting member being at least partially receivable in the groove, the cutting member being selectively operable to maintain the tip member and the shaft member in a fixed relationship to one another.

In accordance with one aspect of this embodiment, the shaft member includes an area defining a slot formed at a distal portion thereof.

In accordance with one aspect of this embodiment, the cutting member includes an engagement member selectively operable to engage the slot of the shaft member.

In accordance with one aspect of this embodiment, when the cutting member is at least partially received in the groove, the engagement member extends above a top surface of the proximal portion of the tip member.

In accordance with one aspect of this embodiment, the tip member is formed of a transparent material.

In accordance with one aspect of this embodiment, the proximal portion of the tip member is conically shaped.

In accordance with one aspect of this embodiment, the shaft member or the cutting member is comprised of a metallic material.

In accordance with one aspect of this embodiment, the proximal portion of the tip member has an outer diameter less than an internal diameter of the bore of the shaft member.

In accordance with one aspect of this embodiment, the proximal portion of the tip member has an outer diameter less than an external diameter of the distal portion of the shaft member so as to form an area defining a shoulder portion therebetween.

In accordance with one aspect of this embodiment, the shoulder portion is selectively operable to abut against the distal end of the shaft member so as to prevent further movement of the proximal portion of the tip member into the bore.

In accordance with a second embodiment of the present invention, a trocar system is provided, comprising:

a shaft member having a bore formed at a distal end thereof, the shaft member having an area defining a slot formed at a distal portion thereof;

a tip member being formed of a transparent material and having a proximal portion and a distal portion, the tip member having an area defining a groove extending along a peripheral surface thereof, the proximal portion being at least partially receivable in the bore; and

a cutting member being at least partially receivable in the groove, the cutting member having an engagement member selectively operable to engage the slot of the shaft member, the engagement member being selectively operable to maintain the tip member and the shaft member in a fixed relationship to one another.

In accordance with one aspect of this embodiment, when the cutting member is at least partially received in the groove, the engagement member extends above a top surface of the proximal portion of the tip member.

In accordance with one aspect of this embodiment, the proximal portion of the tip member is conically shaped.

In accordance with one aspect of this embodiment, the shaft member or the cutting member is comprised of a metallic material.

In accordance with one aspect of this embodiment, the proximal portion of the tip member has an outer diameter less than an internal diameter of the bore of the shaft member.

In accordance with one aspect of this embodiment, the proximal portion of the tip member has an outer diameter less than an external diameter of the distal portion of the shaft member so as to form an area defining a shoulder portion therebetween.

In accordance with one aspect of this embodiment, the shoulder portion is selectively operable to abut against the distal end of the shaft member so as to prevent further movement of the proximal portion of the tip member into the bore.

In accordance with a third embodiment of the present invention, a trocar system is provided, comprising:

a shaft member having an area defining a throughbore formed therethrough, the throughbore extending from a proximal opening to a distal opening of the shaft member, the shaft member having a cutting member formed at, and extending away from, a distal portion thereof;

a tip member having a proximal portion and a distal portion, the tip member having an area defining a groove extending along a peripheral surface thereof, the tip portion being receivable in the proximal opening of the through bore such that the distal portion of the tip portion is selectively operable to extend at least partially past the distal opening; and

wherein the cutting member is at least partially receivable in the groove, the cutting member being selectively operable to maintain the tip member and the shaft member in a fixed relationship to one another.

In accordance with one aspect of this embodiment, the tip member is formed of a transparent material.

In accordance with one aspect of this embodiment, the proximal portion of the tip member is conically shaped.

In accordance with one aspect of this embodiment, the shaft member or the cutting member is comprised of a metallic material.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1 illustrates a perspective view of a trocar system showing the underside of the handle portion, in accordance with a first embodiment of the present invention;

FIG. 2 illustrates a perspective view of a trocar system, in accordance with a second embodiment of the present invention;

FIG. 3 illustrates a perspective view of a trocar system showing a partial cutaway view of the shaft portion, in accordance with a third embodiment of the present invention;

FIG. 4 illustrates a perspective view of a hollow top tip portion, in accordance with a fourth embodiment of the present invention;

FIG. 5 illustrates a side view of a tip portion, in accordance with a fifth embodiment of the present invention;

FIG. 6 illustrates a perspective view of a solid top tip portion, in accordance with a sixth embodiment of the present invention;

FIG. 7 illustrates a perspective view of a cutting member, in accordance with a seventh embodiment of the present invention;

FIG. 8 illustrates a detail view of a tip portion/cutting member tandem or assembly, in accordance with an eighth embodiment of the present invention;

FIG. 9 illustrates a cutaway detail view of a tip portion/cutting member tandem or assembly, in accordance with a ninth embodiment of the present invention;

FIG. 10 illustrates a perspective view of a shaft portion, in accordance with a tenth embodiment of the present invention;

FIG. 11 illustrates a detail view of a slot formed in a shaft portion, in accordance with an eleventh embodiment of the present invention;

FIG. 12 illustrates a top perspective view of a handle portion, in accordance with a twelfth embodiment of the present invention;

FIG. 13 illustrates a bottom perspective view of a handle portion, in accordance with a thirteenth embodiment of the present invention;

FIG. 14 illustrates a perspective view of a cannula system, in accordance with a fourteenth embodiment of the present invention;

FIG. 15 illustrates a perspective view of a combined trocar/cannula system, in accordance with a fifteenth embodiment of the present invention;

FIG. 16 illustrates a perspective view of a first alternative hollow top tip portion, in accordance with a sixteenth embodiment of the present invention;

FIG. 17 illustrates a perspective view of a first alternative cutting member, in accordance with a seventeenth embodiment of the present invention;

FIG. 18 illustrates a detail view of a first alternative tip portion/cutting member tandem or assembly, in accordance with an eighteenth embodiment of the present invention;

FIG. 19 illustrates a perspective view of a first alternative solid top tip portion, in accordance with a nineteenth embodiment of the present invention;

FIG. 20 illustrates a perspective view of a second alternative hollow top tip portion, in accordance with a twentieth embodiment of the present invention;

FIG. 21 illustrates a perspective view of a second alternative cutting member, in accordance with a twenty-first embodiment of the present invention;

FIG. 22 illustrates a detail view of a second alternative tip portion/cutting member tandem or assembly, in accordance with an twenty-second embodiment of the present invention;

FIG. 23 illustrates a perspective view of a second alternative solid top tip portion, in accordance with a twenty-third embodiment of the present invention;

FIG. 24 illustrates a perspective view of a third alternative hollow top tip portion, in accordance with a twenty-fourth embodiment of the present invention;

FIG. 25 illustrates a perspective view of a third alternative cutting member, in accordance with a twenty-fifth embodiment of the present invention;

FIG. 26 illustrates a detail view of a third alternative tip portion/cutting member tandem or assembly, in accordance with an twenty-sixth embodiment of the present invention;

FIG. 27 illustrates a perspective view of a third alternative solid top tip portion, in accordance with a twenty-seventh embodiment of the present invention;

FIG. 28 illustrates a perspective view of a fourth alternative cutting member, in accordance with a twenty-eighth embodiment of the present invention;

FIG. 29 illustrates a detail view of a fourth alternative tip portion/cutting member tandem or assembly, in accordance with an twenty-ninth embodiment of the present invention;

FIG. 30 illustrates a perspective view of a fifth alternative cutting member, in accordance with a thirtieth embodiment of the present invention; and

FIG. 31 illustrates a detail view of a fifth alternative tip portion/cutting member tandem or assembly, in accordance with a thirty-first embodiment of the present invention.

The same reference numerals refer to the same parts throughout the various Figures.

### DETAILED DESCRIPTION OF THE INVENTION

The following description of the preferred embodiment(s) is merely exemplary in nature and is in no way intended to limit the invention, or uses.

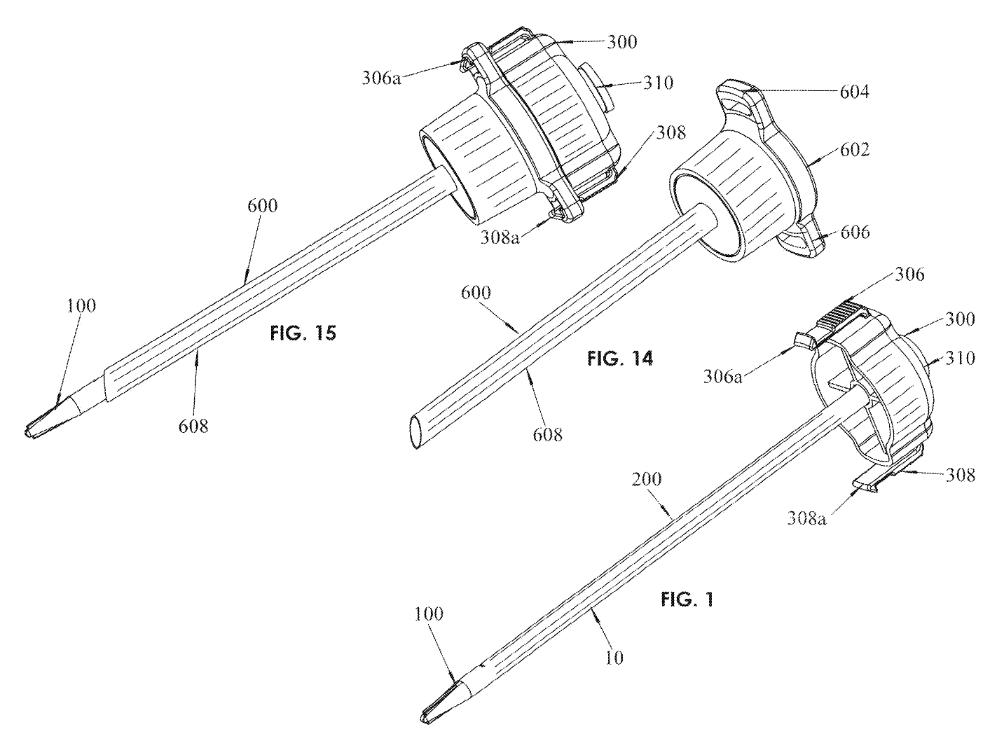

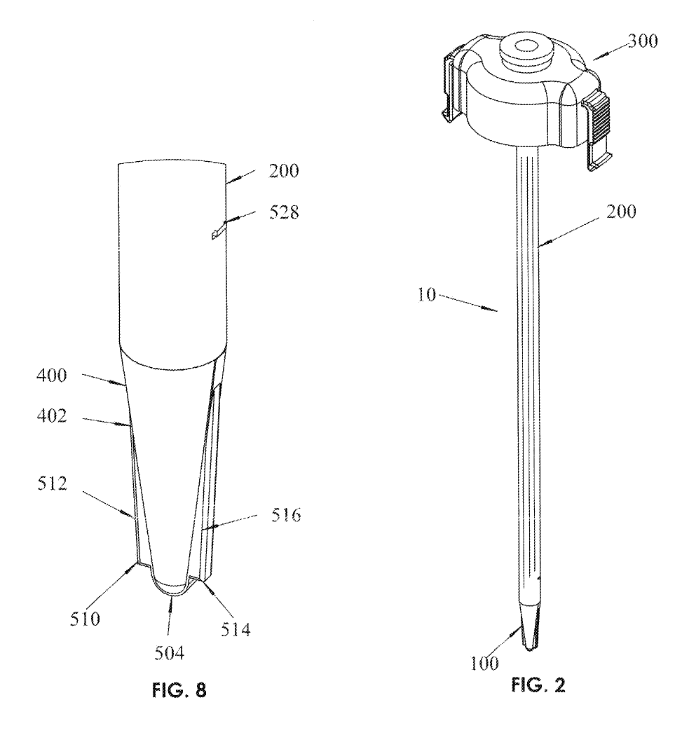

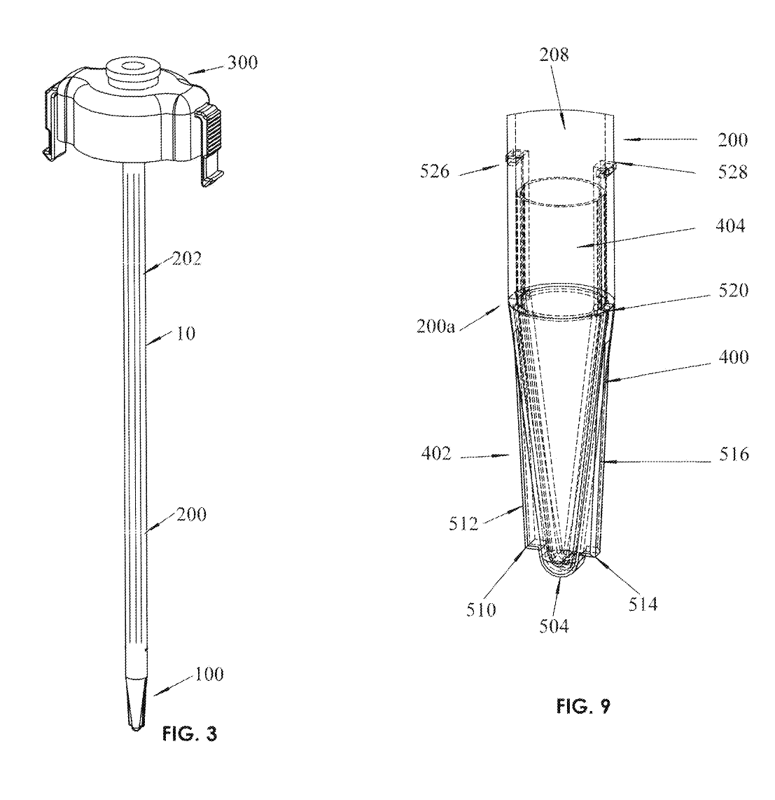

Referring generally to the Figures, and more specifically to FIGS. 1-3, there is shown a trocar system generally at 10. The system 10 primarily includes a tip portion 100, a shaft portion 200 and an optional handle portion 300.

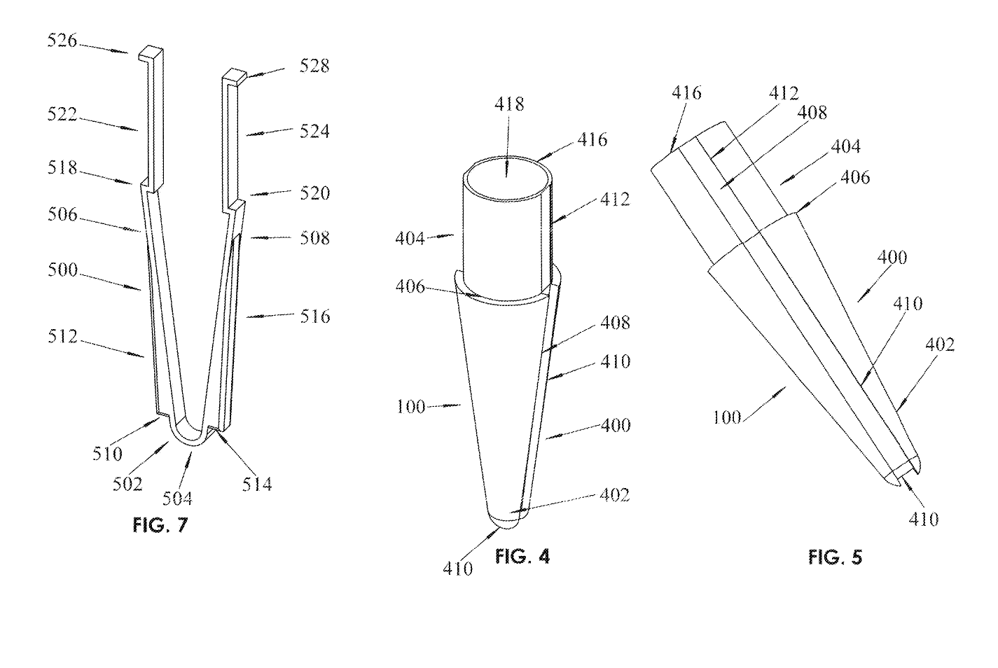

Referring specifically to FIGS. 4 and 5, the tip portion 100 primarily includes a tip member 400 which is preferably comprised of a transparent material, such as but not limited to plastics, such as but not limited to acrylics, polycarbonates, and/or the like, as well as glass and/or the like. By way of a non-limiting example, the chosen material for the tip member 400 should be one that is able to at least partially transmit light (e.g., from any source) therethrough such that the surgeon can adequately visualize the patient's laparoscopic cavity, e.g., with the aid of an illuminating device.

Although the tip portion member 400 is shown as being substantially conical in shape, it should be appreciated that other shapes and/or configurations may be practiced with the present invention.

The tip member 400 may include a lower conical portion 402 and an upper cylindrical portion 404. A stop or shoulder portion 406 is defined at the interface between the lower conical portion 402 and an upper cylindrical portion 404. In this view, at least a portion of the lower conical portion 402 has a larger outer diameter than that of the upper cylindrical portion 404. The lower conical portion 402 includes an area defining a groove 408 (the intended purpose of which will be described herein) extending from the terminus 410 up along the peripheral surface 412 thereof and further extending along the peripheral surface 414 of the upper cylindrical portion 404. The groove 408 terminates at the top portion 416 of the upper cylindrical portion 404. The upper cylindrical portion 404 includes an area defining a bore 418 formed therein, the bore 418 originating at the top portion 416 and extending downwardly towards and/or into the lower conical portion 402, thus providing an essentially hollow tip member 400. The exact depth of the bore 418 is not thought to be critical to the success of the invention; however, it may provide a passageway for an illuminating device (not shown) to be placed near and/or proximate to an internal surface of the lower conical portion 402.

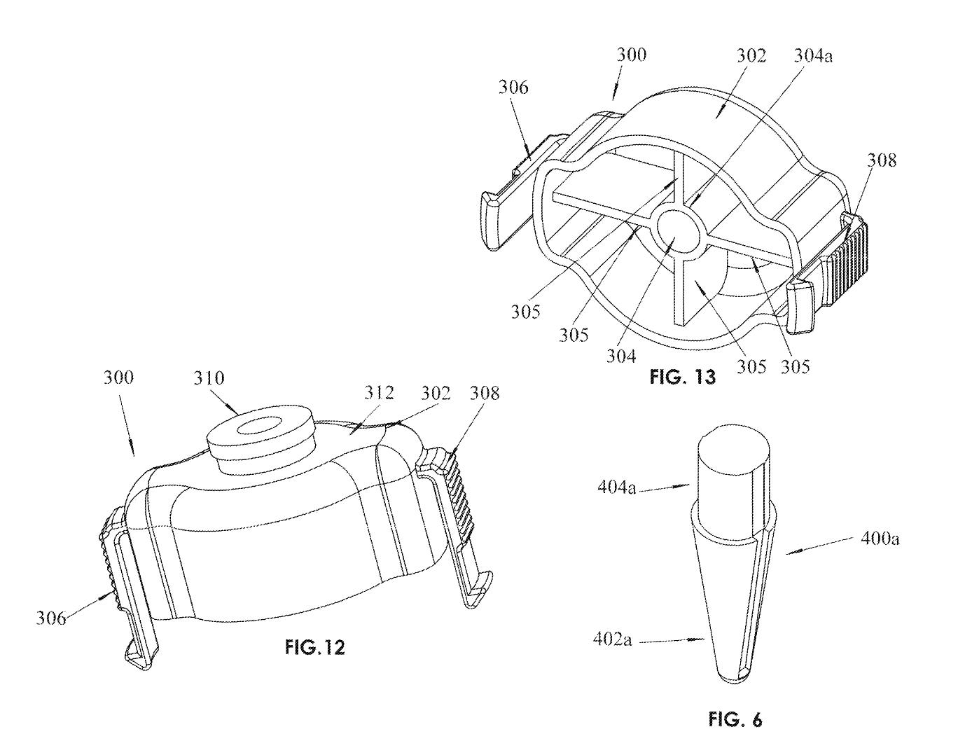

Alternatively, as shown in FIG. 6, the bore 418 can be eliminated altogether, thus resulting in a tip member 400a having a solid lower conical portion 402a and/or upper cylindrical portion 404a. By way of a non-limiting example, the chosen material for the tip member 400a should be one that is able to transmit light (from any source) therethrough such that the surgeon can adequately visualize the patient's laparoscopic cavity, e.g., with the aid of an illuminating device.

Referring specifically to FIGS. 7-9, a cutting member 500 is shown. By way of a non-limiting example, the cutting member 500 may be comprised of a metallic material, e.g., a biocompatible metallic material such as but not limited to stainless steel and/or the like.

The cutting member 500 is intended to engage and interoperate with the tip portion 400. The cutting member 500 primarily includes a lower end portion 502 having a rounded or blunted terminus 504. Extending upwardly from the terminus 504 is at least one, and more preferably at least two spaced and opposed arm members 506, 508, respectively. The arm members 506, 508, respectively, may be provided with one or more blade surfaces 510, 512, 514, 516, respectively, formed thereon. One or more of the blade surfaces 510, 512, 514, 516, respectively, may be blunted and/or sharpened. The arm members 506, 508, respectively, extend further upwardly towards a pair of spaced and opposed shoulder surfaces 518, 520, respectively (the intended purpose of which will be described herein). A pair of finger members 522, 524, respectively (the intended purpose of which will be described herein), extend upwardly from the shoulder surfaces 518, 520, respectively. At the terminus of the finger members 522, 524, respectively, a pair of spaced and opposed engagement members 526, 528, respectively (the intended purpose of which will be described herein), extend outwardly therefrom in a perpendicular orientation to the finger members 522, 524, respectively.

By way of a non-limiting example, to engage the cutting member 500 to the tip portion 400, the two components are brought into contact with one another such that the terminus 504 of the cutting member 500 abuts against the terminus 410 whereby the lower end portion 502 is received, at least partially, into the groove 408. In this manner, the arm members 506, 508, respectively, the shoulder surfaces 518, 520, respectively, and finger members 522, 524, respectively, are likewise also received, at least partially, into their respective corresponding portions of the groove 408 as well. In this manner, a tip portion 400/blade member 500 tandem or assembly may be formed.

Once the cutting member 500 is engaged to the tip portion 400, at least a portion of the finger members 522, 524, respectively, preferably extends above the top portion 416 of the upper cylindrical portion 404 such that the engagement members 526, 528, respectively, also extend above the top portion 416 of the upper cylindrical portion 404 and are able to flex either in an inboard (i.e., inwardly) and/or outboard (i.e., outwardly) direction, the intended purpose of which will be described herein.

By way of a non-limiting example, in order to prevent the inadvertent disengagement of the cutting member 500 from the tip portion 400, a chemical and/or mechanical fastening system can be employed. For example, an adhesive, glue, and/or the like can be used to fasten the cutting member 500 to the tip portion 400. The adhesive, glue, and/or the like can be applied to either (or both) the cutting member 500 and/or the tip portion 400 before engagement thereto, or alternatively, the adhesive, glue, and/or the like can be applied to either (or both) the cutting member 500 and/or the tip portion 400 after engagement thereto.

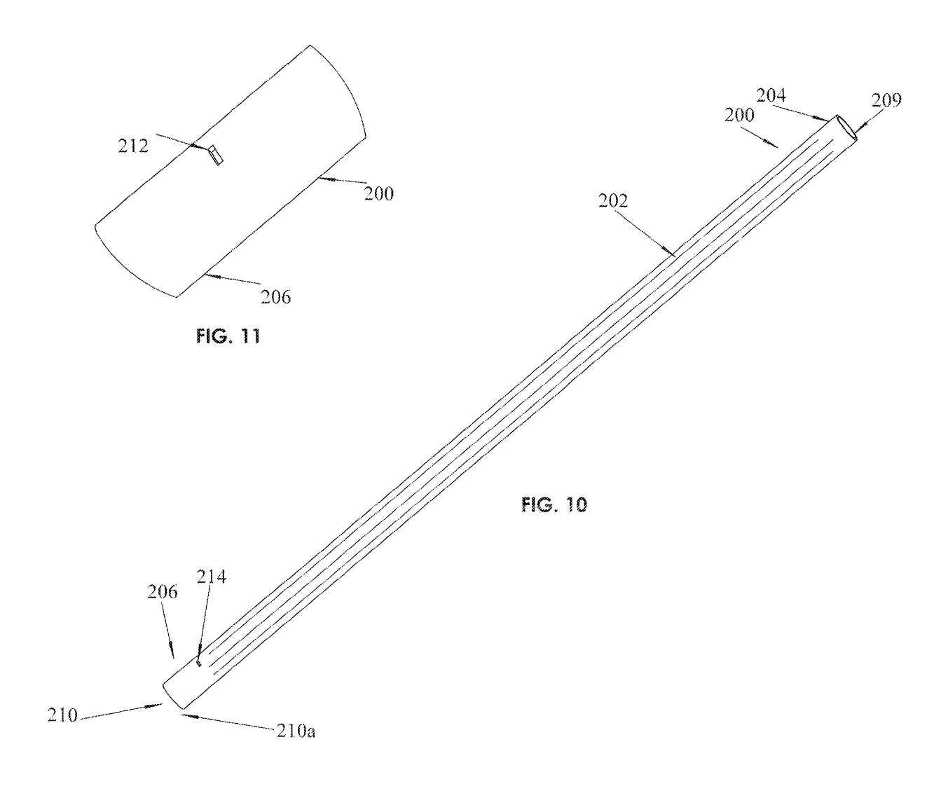

Referring specifically to FIGS. 1-3 and 8-11, the shaft portion 200 primarily includes an elongated, hollow cylindrical tube member 202. By way of a non-limiting example, the shaft portion 200 may be comprised of a metallic material, e.g., a biocompatible metallic material such as but not limited to stainless steel and/or the like.

The tube member 202 includes a proximal portion 204 and a distal portion 206. An area defining a through bore 208 interconnects a proximal opening 209 and a distal opening 210. At the distal portion 206, there may be provided an area defining a pair of spaced and opposed slots 212, 214, respectively (the intended purpose of which will be described herein).

By way of a non-limiting example, to engage the tip portion 400/cutting member 500 tandem or assembly to the distal portion 206 of the shaft portion 200, the two components are brought into contact with one another such that the engagement members 526, 528, respectively, and finger members 522, 524, respectively, are received into the distal opening 210. As the tip portion 400/cutting member 500 tandem and finger members 522, 524, respectively, are further received into the distal opening 210, at least a portion of the upper cylindrical portion 404 is then received into the distal opening 210. Preferably, the upper cylindrical portion 404 has an external diameter less than an internal diameter of the distal opening 210 such that the upper cylindrical portion 404 can relatively easily be received therein. As the upper cylindrical portion 404 is further received into the distal opening 210, the shoulder portion 406 of the upper cylindrical portion 404 abuts against the end portion 210a of the distal opening 210, thus preventing further advancement of the tip portion 400/cutting member 500 tandem relative to the shaft portion 200. At this point, the engagement members 526, 528, respectively, may be proximate to the slots 212, 214, respectively, such that the engagement members 526, 528, respectively, may “snap” into the slots 212, 214, respectively, thus interconnecting the tip portion 400/cutting member 500 tandem or assembly and the shaft portion 200. If the tip portion 400/cutting member 500 tandem or assembly was misaligned relative to the distal opening 210 during the insertion procedure, the tip portion 400/cutting member 500 tandem or assembly can simply be rotated until the engagement members 526, 528, respectively, align with and then snap into the slots 212, 214, respectively. It should be appreciated that the groove 408 preferably has a depth sufficient to permit the flexure of the arm members 506, 508, respectively, and/or finger members 522, 524, respectively, in an inboard direction (e.g., inwardly) before they snap into the slots, 212, 214, respectively.

By way of a non-limiting example, in order to prevent the inadvertent disengagement of the tip portion 400/cutting member 500 tandem or assembly and the shaft portion 200, a chemical and/or mechanical fastening system can be employed. For example, an adhesive, glue, and/or the like can be used to fasten the shoulder portion 406 of the upper cylindrical portion 404 to the end portion 210a of the distal opening 210. The adhesive, glue, and/or the like can be applied to either (or both) the shoulder portion 406 of the upper cylindrical portion 404 to the end portion 210a of the distal opening 210 before engagement thereto, or alternatively, the adhesive, glue, and/or the like can be applied to either (or both) the shoulder portion 406 of the upper cylindrical portion 404 to the end portion 210a of the distal opening 210 after engagement thereto. Furthermore, the engagement members 526, 528, respectively, may be glued to the slots 212, 214, respectively, or alternatively, the engagement members 526, 528, respectively, may be welded to the slots 212, 214, respectively.

Referring specifically to FIGS. 1-3, 12 and 13, the handle portion 300 primarily includes a hub member 302 having an area defining a centrally located through bore 304 formed therein. At least one wall member 305 (and preferably at least two or more) extends from the cylindrical wall 304a of the bore 304 to provide enhanced structural stability to the hub member 302. A pair of spaced and opposed arm members 306, 308, respectively, extends outwardly and downwardly from the hub member 302 (the intended purpose of which will be described herein). A port member 310 may be formed on a top surface 312 of the hub member 302 to allow instrumentation to pass through the port member 310 and the through bore 304. Thus, the port member 310 and the through bore 304 are preferably coaxially aligned with one another. The proximal portion 204 of the tube member 202 is preferably joined to the through bore 304 by any number of methods, including but not limited to chemical and/or mechanical attachment, e.g., gluing, press fit, frictional engagement, and/or the like. Once the tube member 202 is fastened to the through bore 304, the port member 310, the through bore 304, and the through bore 208 are preferably coaxially aligned with one another to allow instrumentation to pass there through.

Referring specifically to FIGS. 14 and 15, the trocar system 10 may be used in conjunction with a cannula system 600. The cannula system 600 primarily includes a handle portion 602 with a pair of spaced and opposed slot members 604, 606, respectively, and an elongated, hollow tube portion 608 extending from the handle portion 602. To engage the trocar system 10 with the cannula system 600, the tip portion 100 is introduced into the top of the through bore 610 of the tube member and advanced therethrough until the tip portion 100 protrudes out through the distal portion 612 of the tube member 600. As this occurs, the arm members 306, 308, respectively, of the hub member 302 engage the slot members 604, 606, respectively, of the handle portion 602 so as to releasably interconnect the two components. Each of the arm members 306, 308, respectively, may be provided with shoulder members 306a, 308a, respectively, abut against the underside surfaces 604a, 606a, respectively, of the slot members 604, 606, respectively, to prevent inadvertent disengagement of the arm members 306, 308, respectively, from the slot members 604, 606, respectively.

Several alternative embodiments are also provided with respect to modifications of the tip portions, cutting members and shaft portions.

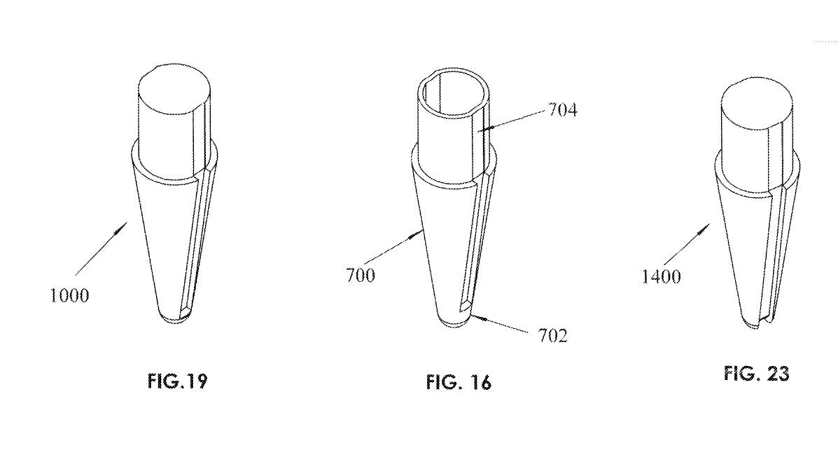

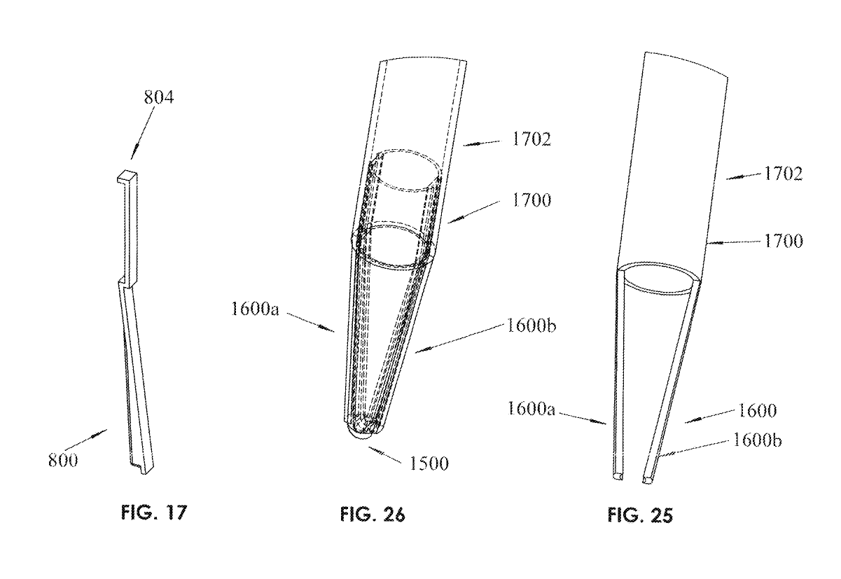

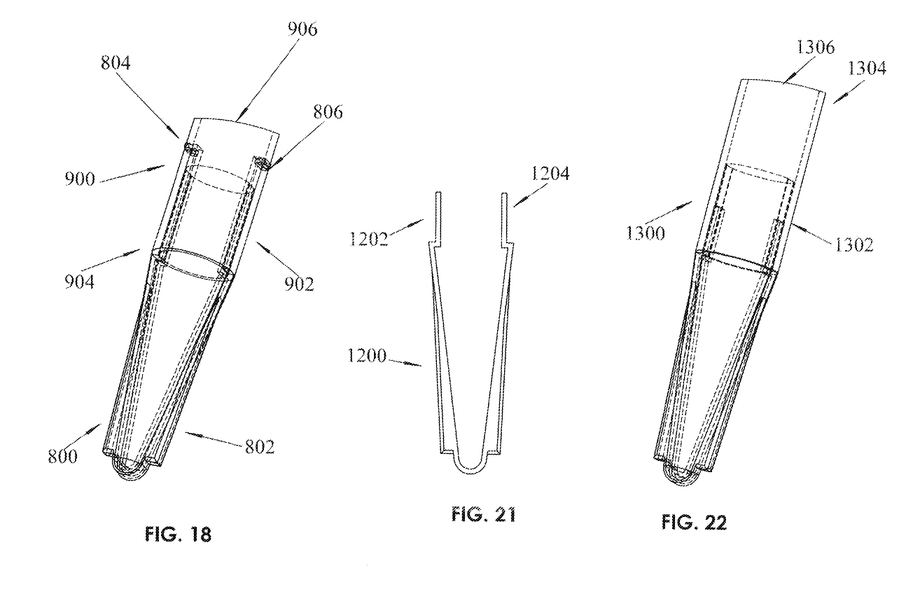

Referring specifically to FIGS. 16-18, there is shown a hollow tip portion 700, wherein the lower terminus portion 702 does not include an area defining a groove 704. Accordingly, two separate cutting members 800, 802, respectively, are needed (as opposed to a single unitary cutting member). As with the previous embodiments, the engagement members 804, 806, respectively, may engage slots 900, 902, respectively, formed on the distal portion 904 of the shaft portion 906. Referring specifically to FIG. 19, a solid version 1000 of the tip portion 700 is shown.

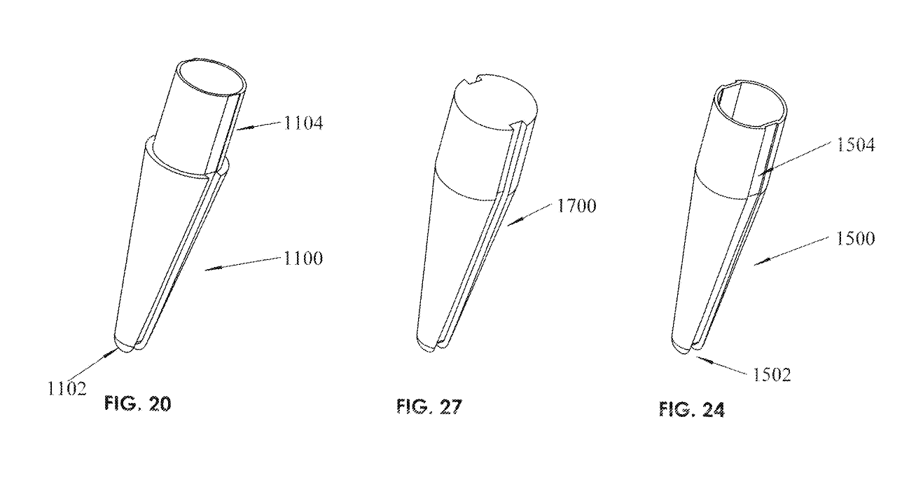

Referring specifically to FIGS. 20-22, there is shown a hollow tip portion 1100, wherein the lower terminus portion 1102 does include an area defining a groove 1104. Accordingly, only one cutting member 1200 is needed (as opposed to two cutting members). Distinct from the previous embodiments, there are no engagement members provided on the cutting member fingers 1202, 1204, respectively, that may engage slots 1300, 1302, respectively, formed on the distal portion 1304 of the shaft portion 1306. In this case, a weld may be formed between the cutting member fingers 1202, 1204, respectively, and the slots 1300, 1302, respectively (alternatively, an adhesive may be used to join the two portions together). Referring specifically to FIG. 23, a solid version 1400 of the tip portion 1100 is shown.

Referring specifically to FIGS. 24-26, there is shown a hollow tip portion 1500, wherein the lower terminus portion 1502 does include an area defining a groove 1504. Additionally, the tip portion 1500 does not include a shoulder portion, thus allowing the tip portion 1500 to be introduced down the proximal portion of the shaft portion such that it drops down the shaft portion and partially exits out through the distal portion 1700. However, in this embodiment a separate cutting member is not provided; but rather, the cutting member 1600 is formed from the distal portion 1700 of the shaft portion 1702 (in this view there are two cutting members 1600a, 1600b, respectively). In this case, the two cutting members 1600a, 1600b, respectively, may be pushed (if needed) into the groove 1504 and secured in place by use of an adhesive and/or the like. Referring specifically to FIG. 27, a solid version 1700 of the tip portion 1500 is shown.

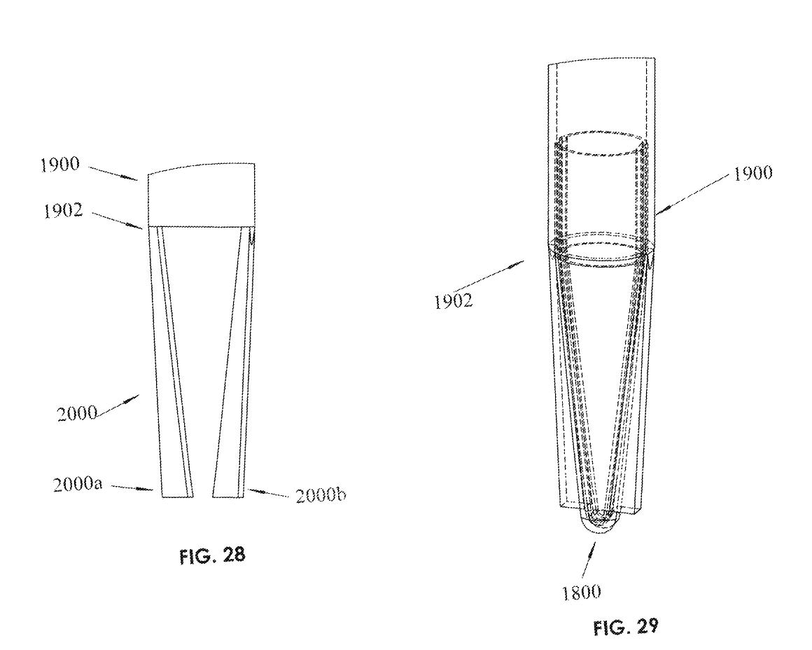

Referring specifically to FIGS. 28 and 29, there is shown a variation of the embodiment depicted in FIGS. 24-27. In this embodiment, the tip portion 1800 does not include a shoulder portion, thus allowing the tip portion 1800 to be introduced down the proximal portion of the shaft portion 1900 such that it drops down the shaft portion 1900 and partially exits out through the distal portion 1902. Again, the cutting member 2000 is formed from the distal portion 1902 of the shaft portion 1900 (in this view there are two cutting members 2000a, 2000b, respectively). However, the cutting members 2000a, 2000b, respectively, include machined edges forming cutting surfaces thereon. Again, in this case, the two cutting members 2000a, 2000b, respectively, may be pushed (if needed) into the groove (not shown) of the tip portion 1800 and secured in place by use of an adhesive and/or the like.

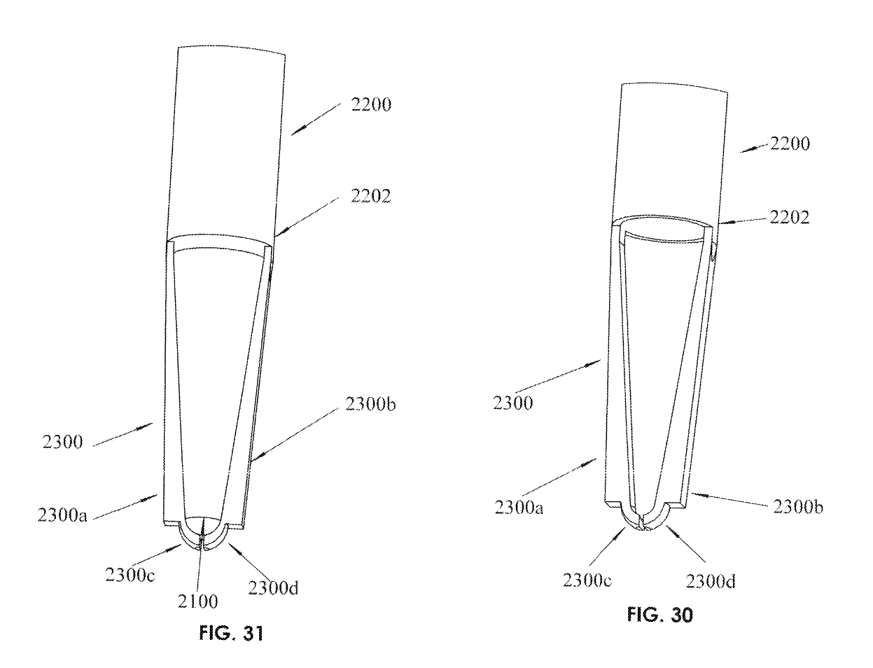

Referring specifically to FIGS. 30 and 31, there is shown a variation of the embodiment depicted in FIGS. 28 and 29. In this embodiment, the tip portion 2100 does not include a shoulder portion, thus allowing the tip portion 2100 to be introduced down the proximal portion of the shaft portion 2200 such that it drops down the shaft portion and partially exits out through the distal portion 2202. Again, the cutting member 2300 is formed from the distal portion 2202 of the shaft portion 2200 (in this view there are two cutting members 2300a, 2300b, respectively). However, the cutting members 2300a, 2300b, respectively, include blunt rounded terminal portions 2300c, 2300d, respectively, formed thereon. Again, in this case, the two cutting members 2300a, 2300b, respectively, may be pushed (if needed) into the groove (not shown) of the tip portion 2100 and secured in place by use of an adhesive and/or the like.

While the invention has been described with reference to an exemplary embodiment, it will be understood by those skilled in the art that various changes can be made and equivalents can be substituted for elements thereof without departing from the scope of the invention. In addition, many modifications can be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A trocar system, comprising:
a shaft member having a bore formed at a distal end thereof;
a tip member having a proximal portion and a distal portion, the tip member having an area defining a groove extending along a peripheral surface thereof, the proximal portion being at least partially receivable in the bore; and
a cutting member being at least partially receivable in the groove, the cutting member being selectively operable to maintain the tip member and the shaft member in a fixed relationship to one another.

2. The system according to claim 1, wherein the shaft member includes an area defining a slot formed at a distal portion thereof.

3. The system according to claim 2, wherein the cutting member includes an engagement member selectively operable to engage the slot of the shaft member.

4. The system according to claim 3, wherein when the cutting member is at least partially received in the groove, the engagement member extends above a top surface of the proximal portion of the tip member.

5. The system according to claim 1, wherein the tip member is formed of a transparent material.

6. The system according to claim 1, wherein the proximal portion of the tip member is conically shaped.

7. The system according to claim 1, wherein when the shaft member or the cutting member is comprised of a metallic material.

8. The system according to claim 1, wherein the proximal portion of the tip member has an outer diameter less than an internal diameter of the bore of the shaft member.

9. The system according to claim 1, wherein the proximal portion of the tip member has an outer diameter less than an external diameter of the distal portion of the shaft member so as to form an area defining a shoulder portion therebetween.

10. The system according to claim 9, wherein the shoulder portion is selectively operable to abut against the distal end of the shaft member so as to prevent further movement of the proximal portion of the tip member into the bore.

11. A trocar system, comprising:
a shaft member having a bore formed at a distal end thereof, the shaft member having an area defining a slot formed at a distal portion thereof;
a tip member being formed of a transparent material and having a proximal portion and a distal portion, the tip member having an area defining a groove extending along a peripheral surface thereof, the proximal portion being at least partially receivable in the bore; and
a cutting member being at least partially receivable in the groove, the cutting member having an engagement member selectively operable to engage the slot of the shaft member, the engagement member being selectively operable to maintain the tip member and the shaft member in a fixed relationship to one another.

12. The system according to claim 11, wherein when the cutting member is at least partially received in the groove, the engagement member extends above a top surface of the proximal portion of the tip member.

13. The system according to claim 11, wherein the proximal portion of the tip member is conically shaped.

14. The system according to claim 11, wherein the shaft member or the cutting member is comprised of a metallic material.

15. The system according to claim 11, wherein the proximal portion of the tip member has an outer diameter less than an internal diameter of the bore of the shaft member.

16. The system according to claim 11, wherein the proximal portion of the tip member has an outer diameter less than an external diameter of the distal portion of the shaft member so as to form an area defining a shoulder portion therebetween.

17. The system according to claim 16, wherein the shoulder portion is selectively operable to abut against the distal end of the shaft member so as to prevent further movement of the proximal portion of the tip member into the bore.

18. A trocar system, comprising:
a shaft member having an area defining a throughbore formed therethrough, the throughbore extending from a proximal opening to a distal opening of the shaft member, the shaft member having a cutting member formed at, and extending away from, a distal portion thereof;
a tip member having a proximal portion and a distal portion, the tip member having an area defining a groove extending along a peripheral surface thereof, the tip portion being receivable in the proximal opening of the through bore such that the distal portion of the tip portion is selectively operable to extend at least partially past the distal opening; and
wherein the cutting member is at least partially receivable in the groove, the cutting member being selectively operable to maintain the tip member and the shaft member in a fixed relationship to one another.

19. The system according to claim 18, wherein the tip member is formed of a transparent material.

20. The system according to claim 18, wherein the proximal portion of the tip member is conically shaped.

21. The system according to claim 18, wherein the shaft member or the cutting member is comprised of a metallic material.
